(19) United States
(12) Patent Application Publication
(10) Pub. No.: US 2026/0053472 A1
(43) Pub. Date: Feb. 26, 2026

(54) ULTRASONIC ELASTICITY IMAGING METHOD AND ULTRASONIC IMAGING APPARATUS

(71) Applicant: SHENZHEN MINDRAY BIO-MEDICAL ELECTRONICS CO., LTD., Shenzhen (CN)

(72) Inventor: Shuangshuang LI, Shenzhen (CN)

(21) Appl. No.: 18/814,256

(22) Filed: Aug. 23, 2024

(30) Foreign Application Priority Data
Aug. 1, 2023 (CN) 202310961081.0

(51) Int. Cl. A61B 8/08; A61B 8/00

(52) U.S. Cl. CPC A61B 8/485; A61B 8/463; A61B 8/5207

(57) ABSTRACT

The ultrasonic imaging apparatus obtains a first shear wave elasticity result and a first strain elasticity result for the external region of a lesion in the target tissue of an object under examination, and a second strain elasticity result for the internal region of the lesion, and determines a quantitative elasticity result for the internal region of the lesion according to the first shear wave elasticity result, the first strain elasticity result, and the second strain elasticity result. Since the shear wave elasticity result and the strain elasticity result for the external region of the lesion and the strain elasticity result for the internal region of the lesion are accurate, the quantitative elasticity result for the internal region of the lesion calculated from the above results is more accurate than the result obtained by directly measuring the shear wave elasticity of the internal region of the lesion.

### CROSS-REFERENCE TO RELATED APPLICATIONS

This application is based on and claims priority to and benefits of Chinese patent Application No. 202310961081.0, filed on Aug. 1, 2023. The entire content of the above-referenced application is incorporated herein by reference.

### TECHNICAL FIELD

The embodiments of the present disclosure relate to medical equipment, and more particularly to ultrasonic elasticity imaging methods and ultrasonic imaging apparatus.

### BACKGROUND OF THE INVENTION

Ultrasonic elastography can non-invasively extract and image the elasticity or softness/hardness information about tissues of a human body, making up for the shortcomings of traditional ultrasonic medical imaging in extraction of mechanical information, and playing an increasingly important role in various clinical practices such as differentiation of malignant from benign tumor, evaluation of the degree of liver fibrosis, angiosclerosis, and muscle and nerve damage.

Shear wave elasticity imaging, a commonly used method for the elastography of clinical examination in liver, may be implemented by generating shear waves in a liver tissue based on acoustic radiation forces, then detecting their propagation by ultrasound, and calculating physical quantities including propagation velocity of the shear waves and Young's modulus for imaging, thereby quantitatively measuring the elasticity parameters of the liver tissue. This method is a quantitative imaging one, which has significant advantages in evaluating the progression and severity of chronic liver fibrosis, so it is widely used and welcome in clinical practice in liver.

However, due to the fact that shear wave sources generated in tissues are often only at a depth of micrometers, such as only 10 microns, the penetration thereof is limited for large-area and high-hardness lesions (such as malignant cancer). For focal liver tumors, the propagation of shear waves is often difficult to penetrate it, resulting in poor performance of shear wave elastography. This may lead to inaccurate results of shear wave measurement for the inner of the lesions and inexact presentation of the morphology and boundaries of the lesions.

### SUMMARY OF THE INVENTION

The embodiments of the present disclosure provide ultrasonic elasticity imaging methods and ultrasonic imaging apparatus for improving the accuracy and reliability of shear wave elasticity results for the internal region of tissue lesions.

In one embodiment, an ultrasonic elasticity imaging method is provided, which may include:
obtaining an ultrasonic image of a target tissue, and determining an internal region of a lesion and an external region of the lesion of the target tissue according to the ultrasonic image;
transmitting first acoustic radiation force impulses to the external region of the lesion, so as to cause a tissue in the external region of the lesion to generate displacement or strain and generate first shear waves propagating in the external region of the lesion;
transmitting first ultrasonic waves at different times to the external region of the lesion for detecting changes in the displacement or strain of the tissue in the external region of the lesion and tracking the first shear waves propagating in the external region of the lesion, and receiving echoes of the first ultrasonic waves to obtain first ultrasonic echo signals;
obtaining a first strain elasticity result for the external region of the lesion according to the first ultrasonic echo signals;
obtaining a first shear wave elasticity result for the external region of the lesion according to the first ultrasonic echo signals;
transmitting second acoustic radiation force impulses to the internal region of the lesion, so as to cause a tissue in the internal region of the lesion to generate displacement or strain;
transmitting second ultrasonic waves at different times to the internal region of the lesion for detecting changes in the displacement or strain of the tissue in the internal region of the lesion, and receiving echoes of the second ultrasonic waves to obtain second ultrasonic echo signals;
obtaining a second strain elasticity result for the internal region of the lesion according to the second ultrasonic echo signals;
obtaining a quantitative elasticity result for the internal region of the lesion according to the first strain elasticity result, the first shear wave elasticity result and the second strain elasticity result; and
displaying the ultrasonic image and the quantitative elasticity result.

In one embodiment, an acoustic radiation force generated by the first acoustic radiation force impulses on a first target position in the external region of the lesion and an acoustic radiation force generated by the second acoustic radiation force impulses on a second target position in the internal region of the lesion are identical, or a difference therebetween is within a predetermined range, where the first target position is a position corresponding to the first strain elasticity result, and the second target position is a position corresponding to the second strain elasticity result.

In one embodiment, a value of a parameter of the first acoustic radiation force impulses are identical to a value of a parameter of the second acoustic radiation force impulses, and a depth of the first target position in the external region of the lesion is identical to a depth of the second target position in the internal region of the lesion.

In one embodiment, said transmitting first acoustic radiation force impulses to the external region of the lesion includes: transmitting the first acoustic radiation force impulses with a parameter value thereof being a first target value to the external region of the lesion; and said transmitting second acoustic radiation force impulses to the internal region of the lesion includes: transmitting the second acoustic radiation force impulses with a parameter value thereof being a second target value to the internal region of the lesion; where the second target value is determined according to the depth of the first target position in the external region of the lesion, the depth of the second target position in the internal region of the lesion, and the first target value.

In one embodiment, the parameter of the first acoustic radiation force impulses and/or the parameter of the second acoustic radiation force impulses includes at least one of a pulse length, a pulse waveform, an emission frequency, an emission aperture and a focusing intensity.

In one embodiment, said transmitting second acoustic radiation force impulses to the internal region of the lesion includes: transmitting the second acoustic radiation force impulses to one or more target positions in the internal region of the lesion, so as to cause tissue(s) corresponding to the one or more target positions to generate displacement or strain; said transmitting second ultrasonic waves at different times to the internal region of the lesion includes: transmitting the second ultrasonic waves at different times to the one or more target positions in the internal region of the lesion so as to detect changes in the displacement or strain of the tissue(s) corresponding to the one or more target positions; said receiving echoes of the second ultrasonic waves to obtain second ultrasonic echo signals includes: receiving the echoes of the second ultrasonic waves at different times from the one or more target positions to obtain the second ultrasonic echo signals.

In one embodiment, said obtaining a second strain elasticity result for the internal region of the lesion according to the second ultrasonic echo signals includes: obtaining the second strain elasticity result for the one or more target positions according to the second ultrasonic echo signals from the one or more target positions.

In one embodiment, said determining a quantitative elasticity result for the internal region of the lesion according to the first strain elasticity result, the first shear wave elasticity result and the second strain elasticity result includes:
determining the quantitative elasticity result for the one or more target positions in the internal region of the lesion according to the first strain elasticity result, the first shear wave elasticity result, and the second strain elasticity result for the one or more target positions in the internal region of the lesion.

In one embodiment, after obtaining the quantitative elasticity result for the target positions in the internal region of the lesion, the method further includes:
making statistics on the quantitative elasticity result for the target positions in the internal region of the lesion, and displaying a statistical result therefrom, the statistical result including one or more of a median, a mean, a maximum, a minimum, and a standard deviation.

In one embodiment, said displaying the ultrasonic image and the quantitative elasticity result includes:
displaying the ultrasonic image, mapping the quantitative elasticity result for the target positions in the internal region of the lesion into a quantitative elasticity image with a target display effect, and displaying the quantitative elasticity image with the target display effect, the target display effect including any one or more of display effects of grayscale, pseudo-color, and color.

In one embodiment, the target tissue includes a liver tissue, the external region of the lesion includes a diffuse disease region outside the lesion or a non-diseased region outside the lesion of the liver tissue, and the internal region of the lesion includes a focal disease region inside the lesion of the liver tissue.

In one embodiment, the first shear wave elasticity result includes a propagation velocity of the first shear waves, a first Young's modulus or a first shear modulus, and the quantitative elasticity result includes a propagation velocity of second shear waves, a second Young's modulus, or a second shear modulus.

In one embodiment, said obtaining a first shear wave elasticity result for the external region of the lesion according to the first ultrasonic echo signals includes:
determining a duration of the first shear waves propagating from a wave source position to a first reference position according to the first ultrasonic echo signals from the first reference position in the external region of the lesion; and
determining a propagation velocity of the first shear waves in the external region of the lesion according to a distance between the wave source position of the first shear waves and the first reference position and the duration therebetween;

In one embodiment, said obtaining a first shear wave elasticity result for the external region of the lesion according to the first ultrasonic echo signals includes:
determining a time interval between the first shear waves propagating to a first reference position and to a second reference position according to the first ultrasonic echo signals from the first reference position in the external region of the lesion and the first ultrasonic echo signals from the second reference position in the external region of the lesion; and
determining a propagation velocity of the first shear waves in the external region of the lesion according to a distance between the first reference position and the second reference position and the time interval.

In one embodiment, the method further includes:
displaying the first strain elasticity result, the first shear wave elasticity result and the second strain elasticity result;
or, displaying the first shear wave elasticity result;
or, displaying the first shear wave elasticity result, and displaying a comparison result between the first shear wave elasticity result and the quantitative elasticity result.

In one embodiment, determining the external region of the lesion of the target tissue according to the ultrasonic image includes:
determining a location of a first region of interest in the ultrasonic image, taking the first region of interest as the external region of the lesion, and marking the first region of interest in a first mark pattern on the ultrasonic image according to the location of the first region of interest, the first region of interest including a diffuse disease region or a non-diseased region.

In one embodiment, determining the internal region of the lesion of the target tissue according to ultrasonic image includes:
determining a location of a second region of interest in the ultrasonic image, taking the second region of interest as the internal region of the lesion, and marking the second region of interest in a second mark pattern different from the first mark pattern on the ultrasonic image according to the location of the second region of interest, the second region of interest including a focal disease region, and a depth of the diffuse disease region or the non-diseased region relative to a body surface being less than a depth of the focal disease region relative to the body surface.

In one embodiment, said determining the quantitative elasticity result for the internal region of the lesion according to the first strain elasticity result, the first shear wave elasticity result and the second strain elasticity result includes:
determining a first stress result according to the first strain elasticity result and the first shear wave elasticity result; and
determining the quantitative elasticity result according to the first stress result and the second strain elasticity result.

In one embodiment, the first ultrasonic waves include a plurality of sets of ultrasonic waves, the first strain elasticity result is obtained by processing according to the first ultrasonic echo signals corresponding to one set of ultrasonic waves therefrom, and the first shear wave elasticity result is obtained by processing according to the first ultrasonic echo signals corresponding to another set of ultrasonic waves therefrom;
or, the first ultrasonic waves include one set of ultrasonic waves, the first strain elasticity result is obtained by processing according to the first ultrasonic echo signals corresponding to the one set of ultrasonic waves, and the first shear wave elasticity result is obtained by processing according to the first ultrasonic echo signals corresponding to the one set of ultrasonic waves.

In one embodiment, the method further includes:
generating second shear waves propagating in the internal region of the lesion;
transmitting third ultrasonic waves to the internal region of the lesion to track the second shear waves propagating in the internal region of the lesion, and receiving echoes of the third ultrasonic waves to obtain third ultrasonic echo signals;
obtaining a second shear wave elasticity result for the internal region of the lesion according to the third ultrasonic echo signals; and
displaying the second shear wave elasticity result and the quantitative elasticity result, or determining a final elasticity result according to the second shear wave elasticity result and the quantitative elasticity result.

In one embodiment, an ultrasonic elasticity imaging method is provided, which may include:
obtaining an ultrasonic image of a target tissue, the ultrasonic image comprising a first region of interest and a second region of interest of the target tissue;
causing tissues in the first and second regions of interest to generate displacement or strain;
transmitting first ultrasonic waves at different times to the first and second regions of interest for detecting changes in the displacement or strain of the tissues in the first and second regions of interest, and receiving echoes of the first ultrasonic waves to obtain first ultrasonic echo signals;
obtaining a first strain elasticity result for the first region of interest and a second strain elasticity result for the second region of interest according to the first ultrasonic echo signals; generating first shear waves propagating in the first region of interest;
transmitting second ultrasonic waves to the first region of interest for tracking the first shear waves propagating in the first region of interest, and receiving echoes of the second ultrasonic waves to obtain second ultrasonic echo signals;
obtaining a first shear wave elasticity result for the first region of interest according to the second ultrasonic echo signals;
determining a quantitative elasticity result for the second region of interest according to the first strain elasticity result, the second strain elasticity result and the first shear wave elasticity result; and
displaying the ultrasonic image and the quantitative elasticity result.

In one embodiment, there is one or more target tissues, and the tissues in the first and second regions of interest are different regions of a same target tissue, or the tissues corresponding to the first and second regions of interest are different target tissues.

In one embodiment, a depth of a tissue region corresponding to the first region of interest relative to a body surface is less than a depth of a tissue region corresponding to the second region of interest relative to the body surface; or, a hardness of the tissue region corresponding to the first region of interest is less than a hardness of the tissue region corresponding to the second region of interest.

In one embodiment, a driving force that causes the tissue corresponding to the first region of interest to generate displacement or strain is identical to a driving force that causes the tissue corresponding to the second region of interest to generate displacement or strain.

In one embodiment, said displaying the ultrasonic image and the quantitative elasticity result includes:
displaying the ultrasonic image, mapping the quantitative elasticity result for the second region of interest into a quantitative elasticity image with a target display effect, and displaying the quantitative elasticity image with the target display effect; the target display effect including any one or more of display effects of grayscale, pseudo-color, and color.

In one embodiment, the target tissue includes a liver tissue, the first region of interest includes a diffuse disease region outside the lesion or a non-diseased region outside the lesion of the liver tissue, and the second region of interest includes a focal disease region inside the lesion of the liver tissue.

In one embodiment, the method further includes:
displaying the first strain elasticity result, the first shear wave elasticity result and the second strain elasticity result;
or, displaying the first shear wave elasticity result;
or, displaying the first shear wave elasticity result, and displaying a comparison result between the first shear wave elasticity result and the quantitative elasticity result.

In one embodiment, the method further includes:
marking the first region of interest in a first mark pattern on the ultrasonic image according to a location of the first region of interest; and
marking the second region of interest in a second mark pattern on the ultrasonic image according to a location of the second region of interest, the first mark pattern being different from the second mark pattern.

In one embodiment, said determining the quantitative elasticity result for the second region of interest according to the first strain elasticity result, the second strain elasticity result and the first shear wave elasticity result includes:
determining a first stress result according to the first strain elasticity result and the first shear wave elasticity result; and
determining the quantitative elasticity result according to the first stress result and the second strain elasticity result.

In one embodiment, the method further includes:
generating second shear waves propagating in the second region of interest;
transmitting third ultrasonic waves to the second region of interest so as to track the second shear waves propagating in the second region of interest, and receiving echoes of the third ultrasonic waves to obtain third ultrasonic echo signals;
obtaining a second shear wave elasticity result for the second region of interest according to the third ultrasonic echo signals; and
comparing and displaying the second shear wave elasticity result and the quantitative elasticity result, or determining a final elasticity result according to the second shear wave elasticity result and the quantitative elasticity result.

In one embodiment, an ultrasonic imaging apparatus is provided, which may include: an ultrasonic probe;
a transmitting circuit configured to excite the ultrasonic probe to transmit ultrasonic waves to a target tissue of an object under examination;
a receiving circuit configured to control the ultrasonic probe to receive echoes of the ultrasonic waves from the target tissue to obtain ultrasonic echo signals;
a display; and
a processor configured to:
process the ultrasonic echo signals to obtain an ultrasonic image of the target tissue, and determine an internal region of a lesion and an external region of the lesion of the target tissue according to the ultrasonic image;
control the transmitting circuit to excite the ultrasonic probe to transmit first acoustic radiation force impulses to the external region of the lesion, so as to cause a tissue in the external region of the lesion to generate displacement or strain and generate first shear waves propagating in the external region of the lesion;
control the transmitting circuit to excite the ultrasonic probe to transmit first ultrasonic waves at different times to the external region of the lesion for detecting changes in the displacement or strain of the tissue in the external region of the lesion and tracking the first shear waves propagating in the external region of the lesion, and receive echoes of the first ultrasonic waves to obtain first ultrasonic echo signals;
obtain a first strain elasticity result for the external region of the lesion according to the first ultrasonic echo signals;
obtain a first shear wave elasticity result for the external region of the lesion according to the first ultrasonic echo signals;
control the transmitting circuit to excite the ultrasonic probe to transmit second acoustic radiation force impulses to the internal region of the lesion, so as to cause a tissue in the internal region of the lesion to generate displacement or strain;
control the transmitting circuit to excite the ultrasonic probe to transmit second ultrasonic waves at different times to the internal region of the lesion for detecting changes in the displacement or strain of the tissue in the internal region of the lesion, and receive echoes of the second ultrasonic waves to obtain second ultrasonic echo signals;
obtain a second strain elasticity result for the internal region of the lesion according to the second ultrasonic echo signals;
obtain a quantitative elasticity result for the internal region of the lesion according to the first strain elasticity result, the first shear wave elasticity result and the second strain elasticity result; and
control the display to display the ultrasonic image and the quantitative elasticity result.

It can be seen from the above technical schemes that the embodiments of the present disclosure may have the following advantages:

The ultrasonic imaging apparatus can obtain separately a first shear wave elasticity result and a first strain elasticity result for the external region of a lesion in the target tissue of an object under examination, obtain a second strain elasticity result for the internal region of the lesion in the target tissue, and determine a quantitative elasticity result for the internal region of the lesion according to the first shear wave elasticity result, the first strain elasticity result and the second strain elasticity result. Since the shear wave elasticity result and the strain elasticity result for the external region of the lesion, as well as the strain elasticity result for the internal region of the lesion are accurate, the quantitative elasticity result for the internal region of the lesion calculated from the above results is more accurate than the result obtained by directly measuring the shear wave elasticity of the internal region of the lesion, and the morphology and boundary of the lesion can be presented more accurately.

### BRIEF DESCRIPTION OF THE DRAWINGS

FIG. 1 is a schematic block diagram of the structure of an ultrasonic imaging apparatus in some embodiments of the present disclosure;

FIG. 2 is a schematic flowchart of an ultrasonic elasticity imaging method in some embodiments of the present disclosure;

FIG. 3 is a schematic diagram of exemplary locations of the internal region of a lesion and the external region of the lesion in an ultrasonic image in some embodiments of the present disclosure;

FIG. 4 is a schematic diagram of exemplary location of a target tissue for calculating shear wave elasticity results and strain results in some embodiments of the present disclosure;

FIG. 5 is a schematic diagram of a mark pattern for marking the internal region of a lesion in an ultrasonic image in some embodiments of the present disclosure;

FIG. 6 is a schematic diagram of a mark pattern for marking the internal region of a lesion and the external region of the lesion in an ultrasonic image in some embodiments of the present disclosure; and

FIG. 7 is another schematic flowchart of an ultrasonic elasticity imaging method in some embodiments of the present disclosure.

### DETAILED DESCRIPTION

The embodiments of the present disclosure provide ultrasonic elasticity imaging methods and ultrasonic imaging apparatus for improving the accuracy and reliability of shear wave elasticity results for the internal region of tissue lesions.

The terms “first”, “second”, “third”, “fourth”, etc. (if any) in the specification and claims of the present disclosure and in the drawings attached above are used to distinguish similar objects and are not necessarily used to describe a specific order or sequence. It should be understood that the data used in this way are interchangeable where appropriate so that the embodiments described herein can be implemented in an order other than what is illustrated or described here. In addition, the terms “including” and “having”, as well as any variations thereof, are intended to cover non- exclusive inclusions, for example, a process, method, system, product or device comprising a series of steps or units need not be limited to those steps or units that are clearly listed. Instead, it may include other steps or units that are not clearly listed or are inherent to these processes, methods, products or equipment.

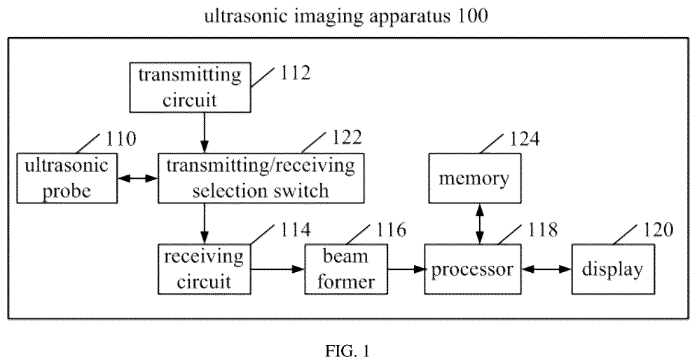

Please refer to FIG. 1, an ultrasonic imaging apparatus 100 in some embodiments may include:
an ultrasonic probe 110, a transmitting circuit 112, a receiving circuit 114, a processor 118 and a display 120. In addition, a transmitting/receiving selection switch 122, a beam former 116 and a memory 124 may also be included.

The ultrasonic probe 110 may be a 2D ultrasonic probe, a 3D ultrasonic probe, or any probe used for ultrasonic inspection and measurement. The acoustic head part of the ultrasonic probe 110 may be an array composed of multiple array elements, such as the array elements arranged in a row to form a line array, or in a two-dimensional matrix to form a plane array. The array elements may also be arranged to form a convex array. The array elements may be used to transmit ultrasonic beams according to excitation of electrical signals, or to convert received ultrasonic echoes into electrical signals. Accordingly, each array element may be used to realize the mutual conversion of the electrical pulse signals and the ultrasonic beams, thereby transmitting ultrasonic waves to a target tissue of a human body and receiving echoes of the ultrasonic waves reflected by the tissue.

The transmitting circuit 112 may be configured to generate transmitting sequences according to the control sent by a transmitting control unit of the processor 118. The transmitting sequences may be configured to control part or all of the array elements to transmit ultrasonic waves to biological tissues.

The receiving circuit 114 may be configured to receive electrical signals of the ultrasonic echoes from the ultrasonic probe 110 to obtain ultrasonic echo signals, and send the ultrasonic echo signals to the beam former 116.

The beam former 116 may be configured to process the signals output by the receiving circuit 114, including delay, weighted summation, and beam forming. Due to the different distances between ultrasonic wave receiving points in the tissue under examination and receiving array elements, the channel data of the same receiving point output from different receiving array elements may have delay differences; accordingly, it is necessary to carry out delay processing to align the phases, weight and sum the different channel data of the same receiving point to obtain beam-formed data.

The processor 118 may be connected with the beam former 116 and may mainly be configured to process, including detecting, signal enhancing, data converting and logarithm compressing, the beam-formed data to generate an ultrasonic image. The ultrasonic image obtained by the processor 118 may be displayed on the display 120 or stored in the memory 124.

Optionally, the processor 118 may be at least one of an application specific integrated circuit (ASIC), a digital signal processor (DSP), a digital signal processing device (DSPD), a programmable logic device (PLD), a field programmable gate array (FPGA), a central processing unit (CPU), a controller, a micro-controller, or a micro-processor, allowing the processor 118 to control other components in the ultrasonic imaging apparatus 100 to perform the ultrasonic imaging steps described in various embodiments disclosed in this specification. The processor 118 may be a component or a general term for control units and processing units in the ultrasonic imaging apparatus capable of controlling other components of the ultrasonic imaging apparatus to perform various functions in various embodiments.

The display 120 may be connected with the processor 118, may be a touch screen or a liquid crystal display, etc. Alternatively, the display 120 may be an independent display such as a liquid crystal display or a television set independent of the ultrasonic imaging apparatus 100. Alternatively, the display 120 may be the display of an electronic device such as a smartphone or tablet, etc. The number of the display 120 may be one or more.

In addition to the above structure, the ultrasonic imaging apparatus 100 may also include a human-machine interaction device. Specifically, the human-machine interaction device may be a display 120, such as all the functions of the human-machine interaction device integrated into the display 120, accordingly, the display 120 can also provide a graphical interface for user interaction when displaying ultrasonic images. One or more controlled objects may be arranged on the graphical interface to provide users with the ability to use the human-machine interaction device to input operation instructions so as to control these controlled objects, thereby executing corresponding control operations. For example, when an icon is displayed on a graphical interface, the icon can be manipulated by a human-machine interaction device to perform specific functions, such as repositioning images and/or zooming in on specific areas.

The human-machine interaction device may also be other human-machine interaction devices besides the display 120. For example, the human-machine interaction device may include an input device for detecting information input by a user. The input information may be such as instructions for editing and marking ultrasonic images, or other types of instructions. The input device may include a keyboard, a scroll wheel, a trackball, and a mobile input device (such as a mobile device with a touch screen, a cell phone, etc.), a multifunctional knob, and any combination thereof. The human-machine interaction device may also include an output device such as a printer.

The ultrasonic imaging apparatus 100 mentioned above may also include a memory 124 for storing instructions for processing execution, for storing received ultrasonic echo signals, for storing ultrasonic image data, and so on. The memory 124 may be a volatile memory, such as a random access memory (RAM), or be a non-volatile memory, such as a read only memory (ROM), a flash memory, a hard disk drive (HDD), or a solid state drive (SSD); or a combination thereof; and may provide instructions and data to the processor.

It should be understood that the components included in the ultrasonic imaging apparatus 100 shown in FIG. 1 are illustrative only and may include more or fewer components, which are not limited in the present disclosure.

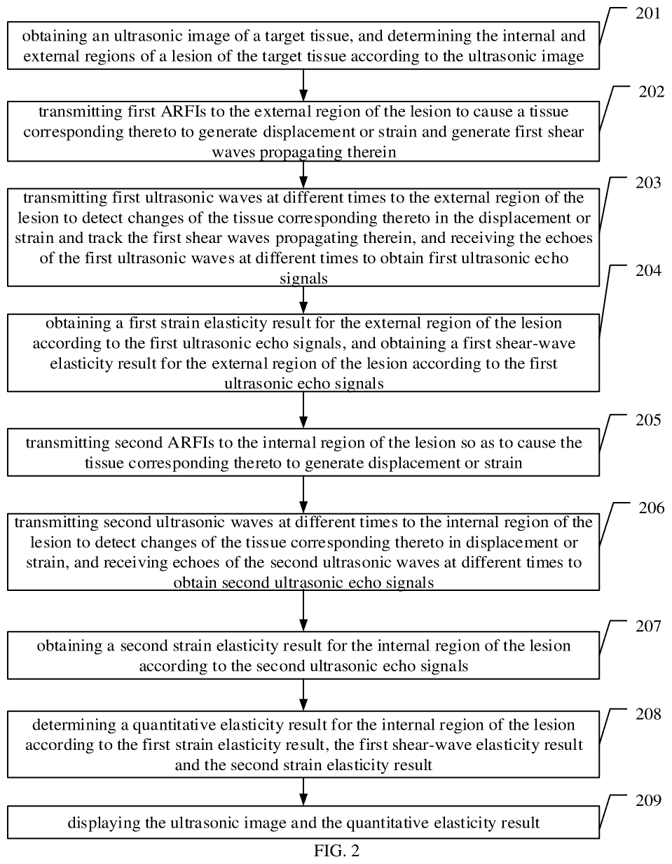

The embodiments and implementations thereof of an ultrasonic elasticity imaging method executed by the ultrasonic imaging apparatus may further be described in detail below according to the specific structural components and functions thereof of the ultrasonic imaging apparatus mentioned above. Please refer to FIG. 2, the ultrasonic elasticity imaging method in some embodiments of the present disclosure may include the following steps:
Step 201: obtaining an ultrasonic image of a target tissue, and determining the internal region of a lesion and the external region of the lesion of the target tissue according to the ultrasonic image;

In some embodiments, the ultrasonic imaging apparatus may, under a scanning operation input by a user, perform ultrasound scanning on the target tissue of an object under examination to obtain the ultrasonic image of the target tissue, and may determine the internal and external regions of the lesion of the target tissue according to the ultrasonic image, respectively.

The target tissue may be any organ tissue of the object under examination, such as a liver tissue. The determination of the internal and external regions of the lesion in the ultrasonic image by the ultrasonic imaging apparatus may be implemented: by using a pre-trained neural network model for image recognition to identify the internal and external regions of the lesion; or by marking the internal and external regions of the lesion on the ultrasonic image by a user so that the ultrasonic imaging apparatus can determine the two regions according to user instructions; or by a combination thereof, that is, the ultrasonic imaging apparatus may automatically determine the two regions, and the regions determined by the ultrasonic imaging apparatus may be manually adjusted by users if they are not satisfied with the determined regions. The way to determine the internal region of the lesion and the external region of the lesion in the ultrasonic image by the ultrasonic imaging apparatus is not limited in the embodiments.

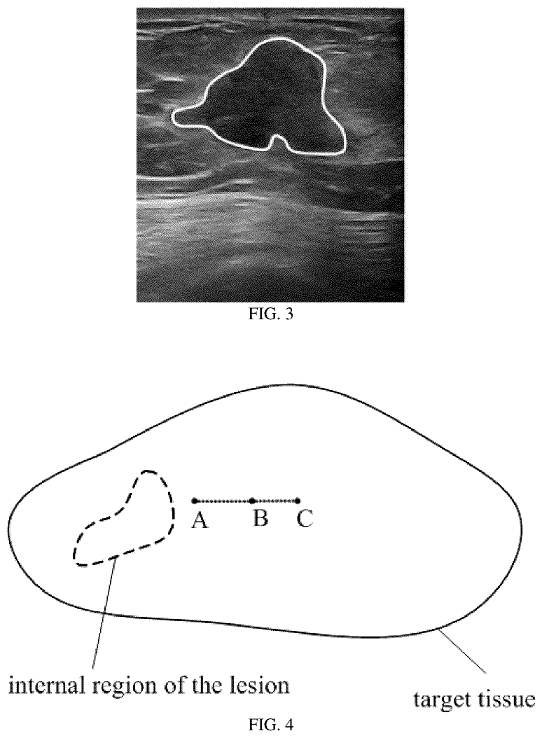

For example, as shown in FIG. 3, the internal region of the lesion in the ultrasonic image is a region surrounded by white lines, and the external region of the lesion is the other region outside the region surrounded by white lines. These two regions may be determined automatically by the ultrasonic imaging apparatus or determined by the ultrasonic imaging apparatus according to user instructions.
Step 202: transmitting first acoustic radiation force impulses to the external region of the lesion to cause a tissue corresponding to external region of the lesion to generate displacement or strain and generate first shear waves propagating in the external region of the lesion;

The ultrasonic imaging apparatus may, according to the internal region of the lesion and the external region of the lesion in the ultrasonic image, further determine the internal region of the lesion and the external region of the lesion in the target tissue, and transmit first acoustic radiation force impulses (ARFIs) such as focused ultrasonic shear wave pushing pulses (SWPs) to the external region of the lesion in the target tissue. Such impulses may cause the tissue corresponding to external region of the lesion to generate displacement or strain. In addition, based on the principle of acoustic radiation force, a force may also be generated inside the tissue, which may in turn generate the first shear waves propagating in the external region of the lesion.
Step 203: transmitting first ultrasonic waves at different times to the external region of the lesion so as to detect changes in the displacement or strain of the tissue corresponding to external region of the lesion and track the first shear waves propagating in the external region of the lesion, and receiving the echoes of the first ultrasonic waves at different times to obtain first ultrasonic echo signals;
Step 204: obtaining a first strain elasticity result for the external region of the lesion according to the first ultrasonic echo signals, and obtaining a first shear wave elasticity result for the external region of the lesion according to the first ultrasonic echo signals;

Based on the effect of acoustic radiation force, while shear waves are generated in the external region of the lesion, the force also causes displacement or strain of the tissue near the shear wave source (usually within the focus region of the acoustic radiation force impulses). The greater the intensity of the acoustic radiation force impulse, the stronger the acoustic radiation force, and the greater the displacement or strain of the tissue near the shear wave source caused.

Therefore, in order to detect the changes in displacement or strain of the tissue in the external region of the lesion, the ultrasonic imaging apparatus may transmit first ultrasonic waves at different times to the external region of the lesion and receive the echoes of the first ultrasonic waves at different times so as to obtain the first ultrasonic echo signals. The ultrasonic imaging apparatus may also obtain a first strain elasticity result for the external region of the lesion according to the first ultrasonic echo signals. The strain elasticity result may be obtained by pressure elasticity imaging, and a specific imaging method thereof may mainly be implemented by: applying pressure to the target tissue by an ultrasonic probe, obtaining two frames of ultrasonic echo information before and after compression of the target tissue, then calculating the displacement of the corresponding position before and after compression by the ultrasonic echo signals (i.e. the spatial position change of the target tissue at two different times), and calculating the axis gradient of the displacement to obtain the strain elasticity result for each point in the region of the target tissue. The strain elasticity result can intuitively reflect the difference in softness/hardness or elasticity between different tissues. Under the compression of the same external force, the greater the strain, the softer the tissue; and the smaller the strain, the harder the tissue.

For example, acoustic radiation force impulses may be transmitted to a certain location in the external region of the lesion, then ultrasonic waves at different times (e.g. at least two sets of ultrasonic waves with intervals of 1 ms, 2 ms, or 3 ms) may be transmitted to the location, the echo signals of each set of ultrasound waves may be received to determine the motion state of the tissue at the location and the difference thereof before and after may be compared, thereby calculating the strain elasticity result for the location. According to the transformation relationship between physical quantities, the strain elasticity result can usually be obtained by calculating the spatial gradient of displacement.

In addition, the first ultrasonic echo signals may also be configured to track the first shear waves propagating in the external region of the lesion; accordingly, a first shear wave elasticity result for the external region of the lesion may be obtained by processing according to the first ultrasonic echo signals.

For example, for each of the above structures of the ultrasonic imaging apparatus, the transducer in the ultrasonic probe 110 may also be configured to apply acoustic radiation force impulses to the external region of the lesion in the target tissue of the object under examination to generate shear waves. Specifically, during shear wave elasticity imaging, the transducer in the ultrasonic probe may apply acoustic radiation force impulses to the external region of the lesion to generate shear waves; the transmitting circuit 112 may send delay-focused transmitting pulses to the ultrasonic probe 110 by the transmitting/receiving selection switch 122; the ultrasonic probe 110 may be excited by the transmitting pulses to transmit ultrasonic beams to the external region of the lesion to track the shear waves, and may, after a certain delay, receive ultrasonic echoes with tissue information reflected back from the external region of the lesion and convert them into electrical signals again. The receiving circuit 114 may receive the electrical signals generated by the conversion performed by the ultrasonic probe 110 to obtain ultrasonic echo signals, and may send them into the beam former 116; the beam former 116 may process the ultrasonic echo data with focusing delay, weighting, and channel summing, and then send it to the processor 118; and the processor 118 may perform elasticity imaging on the ultrasonic echo signals and calculate the shear wave elasticity result and the strain elasticity result for the external region of the lesion.
Step 205: transmitting second acoustic radiation force impulses to the internal region of the lesion so as to cause the tissue in the internal region of the lesion to generate displacement or strain;

The ultrasonic imaging apparatus may also determine the internal region of the lesion in the target tissue of the object under examination according to the internal region of the lesion in the ultrasonic image, and transmit second acoustic radiation force impulses to the internal region of the lesion in the target tissue. The second acoustic radiation force impulses may cause the tissue in the internal region of the lesion in the target tissue to generate displacement or strain.
Step 206: transmitting second ultrasonic waves at different times to the internal region of the lesion so as to detect changes in displacement or strain of the tissue in the internal region of the lesion, and receiving the echoes of the second ultrasonic waves at different times to obtain second ultrasonic echo signals;
Step 207: obtaining a second strain elasticity result for the internal region of the lesion according to the second ultrasonic echo signals;

Similarly, the ultrasonic imaging apparatus may also transmit the second ultrasonic waves at different times to the internal region of the lesion, receive the echoes of the second ultrasonic waves at different times to obtain the second ultrasonic echo signals, and obtain the second strain elasticity result for the internal region of the lesion according to the second ultrasonic echo signals; wherein the second ultrasonic waves may be configured to detect the changes in displacement or strain of the tissue in the internal region of the lesion.

For example, second acoustic radiation force impulses may be transmitted to the internal region of the lesion so as to cause the displacement or strain of the tissue at any location in the internal region of the lesion, then the second ultrasonic waves at different times (e.g. at least two sets of ultrasonic waves with intervals of 1 ms, 2 ms, 3 ms, etc.) may be transmitted to the location, the echo signals of each set of the ultrasonic waves may be received to determine the motion state of the tissue at the location and the difference thereof before and after may be compared, thereby calculating the strain elasticity result for the location. According to the transformation relationship between physical quantities, the strain elasticity result can usually be obtained by calculating the spatial gradient of displacement.
Step 208: determining a quantitative elasticity result for the internal region of the lesion according to the first strain elasticity result, the first shear wave elasticity result and the second strain elasticity result; and

Due to the fact that the shear wave sources generated within the tissue are often only at a depth of micrometers, such as only ten micrometers, their penetration power is limited for lesions (such as malignant cancer) with large area and high hardness. For focal liver tumors, the propagation of shear waves is often difficult to penetrate it, resulting in inaccurate measurement results of shear waves in the internal region of the lesion. To solve this technical problem, in some embodiments, due to the relatively uniform of the tissue in the external region of the lesion and the relatively small hardness thereof, the detection of shear wave elasticity and strain elasticity is relatively reliable and accurate. The hardness of tumor tissue in the internal region of the lesion is usually high or uneven, and though the generated shear waves cannot propagate well, the strain or displacement inside a focus region of acoustic field directly caused by the acoustic radiation forces is relatively reliable. Therefore, the ultrasonic imaging apparatus may determine the quantitative elasticity result for the internal region of the lesion according to the first strain elasticity result, the first shear wave elasticity result and the second strain elasticity result calculated in the above steps. Due to the fact that the external region of the lesion is generally a diffuse lesion or uniform healthy tissue, shear wave elasticity measurement of the external region of the lesion can usually obtain more accurate measurement results. Therefore, the shear wave elasticity result and the strain elasticity result of the external region of the lesion, as well as the strain elasticity result of the internal region of the lesion, are accurate; and in this way, the shear wave elasticity result for the internal region of the lesion calculated according to the above results are more accurate compared to the result obtained by directly measuring the shear wave elasticity of the internal region of the lesion.

The quantitative elasticity result may be the propagation velocity of the shear waves, Young's modulus, shear modulus and other quantitative elastic results, that is, the quantitative elasticity result may be expressed as the above parameters.
Step 209: displaying the ultrasonic image and the quantitative elasticity result.

The ultrasonic imaging apparatus may display the ultrasonic image of the target tissue of the object under examination, and may display the quantitative elasticity result obtained in the above steps, so that doctors can determine the location of the lesion and the shear wave elasticity results for the lesion based on the ultrasonic image.

In some embodiments, the ultrasonic imaging apparatus may obtain the first shear wave elasticity result and the first strain elasticity result for the external region of the lesion in the target tissue of the object under examination, obtain the second strain elasticity result for the internal region of the lesion in the target tissue, and determine the quantitative elasticity result for the internal region of the lesion according to the first shear wave elasticity result, the first strain elasticity result and the second strain elasticity result. Since the shear wave elasticity result and the strain elasticity result for the external region of the lesion, as well as the strain elasticity result for the internal region of the lesion are accurate, the quantitative elasticity result for the internal region of the lesion calculated from the above results is more accurate than the result obtained by directly measuring the shear wave elasticity of the internal region of the lesion, and the morphology and boundary of the lesion can be presented more accurately.

The shear wave elasticity result may include the Young's modulus result. According to Hooke's law, under a certain stress Stress, the strain elasticity result Strain is inversely proportional to the Young's modulus result E, which can be expressed as Stress=Strain*E. Based on this, in some preferred embodiments, the acoustic radiation force generated by the first acoustic radiation force impulses on the first target position of the external region of the lesion is the same as that generated by the second acoustic radiation force impulses on the second target position of the internal region of the lesion, or the difference between the two acoustic radiation forces is within a predetermined range. The first target position may be the position corresponding to the first strain elasticity result, and the second target position may be the position corresponding to the second strain elasticity result. Therefore, it can be ensured that the stress (i.e., the acoustic radiation force) induced by the first acoustic radiation force impulses on the first target position is the same or approximately the same as the stress induced by the second acoustic radiation force impulses on the second target position. When the stress is the same or approximately the same, that is, the stress OutStress at the external region of the lesion is equal to the stress InStress at the internal region of the lesion, and then according to the formula Stress=Strain*E, it can be obtained:

$$OutStrain * OutE = Instrain ( i ) * InE ( i ) ;$$

where OutStrain refers to the first strain elasticity result, OutE refers to the first shear wave elasticity result, OutStrain*OutE refers to the stress OutStress at the external region of the lesion; Instrain(i) refers to the second strain elasticity result, Ink(i) refers to the quantitative elasticity result, and Instrain(i)*Ink(i) refers to the stress InStress at the internal region of the lesion.

Accordingly, the quantitative elasticity result for the internal region of the lesion can be calculated according to this formula.

In some embodiments, in order to ensure that the acoustic radiation force on the first target position by the first acoustic radiation force impulses is the same as the acoustic radiation force on the second target position by the second acoustic radiation force impulses, or that the difference therebetween is within a predetermined range, and the value(s) of parameter(s) of the first acoustic radiation force impulses may be set to be the same as the value(s) of parameter(s) of the second acoustic radiation force impulses. Moreover, the depth of the first target position in the external region of the lesion is the same as the depth of the second target position in the internal region of the lesion, ensuring that the attenuation degrees of the tissue depth to the energy intensity of the acoustic field generated by the first acoustic radiation force impulses and that generated by the second acoustic radiation force impulses are the same.

For example, when the depth of the first target position in the external region of the lesion is the same as the depth of the second target position in the internal region of the lesion, the first acoustic radiation force impulses and the second acoustic radiation force impulses may be set to be consistent in parameter values such as pulse length, pulse waveform, emission frequency, emission aperture or focusing intensity; thereby ensuring that the acoustic radiation force generated by the first acoustic radiation force impulses on the first target position is the same or approximately the same as the acoustic radiation force generated by the second acoustic radiation force impulses on the second target position.

In addition, another way to ensure that the acoustic radiation force generated by the first acoustic radiation force impulses is the same or approximately the same as the acoustic radiation force generated by the second acoustic radiation force impulses is to send the first acoustic radiation force impulses with the parameter value thereof being a first target value to the external region of the lesion; and determine a second target value corresponding to the first target value according to the depth of the first target position in the external region of the lesion and the depth of the second target position in the internal region of the lesion, and send the second acoustic radiation force impulses with the parameter value thereof being the second target value to the internal region of the lesion. In other words, after determining the parameter value(s) of the first acoustic radiation force impulses, the parameter value(s) of the second acoustic radiation force impulses may be adjusted according to the depth difference between the position at which the first acoustic radiation force impulses acts and the position at which the second acoustic radiation force impulses acts, so that the acoustic radiation force generated by the first acoustic radiation force impulses is the same or approximately the same as that generated by the second acoustic radiation force impulses.

For example, if the depth of the second target position in the internal region of the lesion is greater than the depth of the first target position in the external region of the lesion, when transmitting the second acoustic radiation force impulses, compared with the emission of the first acoustic radiation force impulses, the emission frequency of the second acoustic radiation force impulses may be adjusted lower; or, the pulse length of the second acoustic radiation force impulses may be adjusted higher, or, the number of the array elements in the ultrasonic probe for transmitting the second acoustic radiation force impulses may be increased to improve the signal strength thereof, and so on. Accordingly, by adjusting the parameter value(s) of the second acoustic radiation force impulses to improve the signal strength thereof, it is possible to avoid excessive attenuation of the energy intensity of the acoustic field caused by too deep depth, thereby ensuring that the acoustic radiation forces generated by the two types of acoustic radiation force impulses are the same or approximately the same.

In some embodiments, the calculated quantitative elasticity result for the internal region of the lesion may be the quantitative elasticity result for a single position in the internal region of the lesion, or the quantitative elasticity results for a plurality of positions in the internal region of the lesion. Accordingly, the ultrasonic imaging apparatus can transmit the second acoustic radiation force impulses to one or more target positions in the internal region of the lesion so as to cause the tissue(s) corresponding to the one or more target positions to generate displacement or strain, transmit the second ultrasonic waves at different times to one or more target positions in the internal region of the lesion to detect the changes in displacement or strain of the tissue(s) corresponding to the target position(s), and receive the echoes of the second ultrasonic waves at different times from the one or more target positions to obtain second ultrasonic echo signals, so as to obtain the second strain elasticity result(s) for the one or more target positions according to the second ultrasonic echo signals from the one or more target positions.

After obtaining the second strain elasticity result(s) for the one or more target positions in the internal region of the lesion, the quantitative elasticity result(s) for the one or more target positions in the internal region of the lesion may be determined according to the first strain elasticity result, the first shear wave elasticity result, and the second strain elasticity result for the one or more target positions in the internal region of the lesion.

Specifically, due to the limited range of acoustic radiation force generated by the focus region of the acoustic radiation force impulses (usually less than 0.5 mm*0.5 mm), when the area of the lesion is small, a set of second acoustic radiation force impulses may be transmitted only to the internal region of the lesion or the center thereof, so as to obtain a strain result for the internal region of the lesion. In this way, detection time can be saved and the calculation complexity can be reduced. When the area of the lesion is large and the hardness thereof is uneven, the second acoustic radiation force impulses may be transmitted successively to each different position horizontally or vertically in the internal region of the lesion according to the size of the area of the lesion to obtain a plurality of strain results for a plurality of different local positions, according to which the quantitative elasticity results for the local positions may be calculated, respectively.

After obtaining the quantitative elasticity results for the target positions in the internal region of the lesion, the ultrasonic imaging apparatus may also make statistics on the quantitative elasticity results for the target positions in the internal region of the lesion and display the statistical result. The statistical result may include one or more of median, mean, maximum, minimum, and standard deviation.

In some preferred embodiments, the display of the ultrasonic image and the quantitative elasticity result may include: displaying the ultrasonic image, mapping the quantitative elasticity results for the target positions in the internal region of the lesion into a quantitative elasticity image with a target display effect, and displaying the quantitative elasticity image with the target display effect. The target display effect may include any one or more of display effects of grayscale, pseudo-color, and color.

In some embodiments, the target tissue may be a liver tissue, then the external region of the lesion may include a diffuse disease region outside the lesion and a non-diseased region outside the lesion of the liver tissue, and the internal region of the lesion may include a focal disease region inside the lesion of the liver tissue.

In some preferred embodiments, the first shear wave elasticity result may include the propagation velocity of the first shear waves, Young's modulus, or shear modulus. When the first shear wave elasticity result is the propagation velocity of the first shear waves, one way of calculation may be to determine a duration of the propagation of the first shear waves from the wave source position to the first reference position according to the first ultrasonic echo signals from the first reference position in the external region of the lesion, and determine the propagation velocity of the first shear waves in the external region of the lesion according to the distance between the wave source position of the first shear waves and the first reference position and the duration.

The duration of the propagation of the first shear waves from the wave source position to the first reference position may be a time interval between the generation of the first shear waves and the propagation of the first shear waves to the first reference position, wherein the generation of the first shear waves may be the time during which the ultrasonic imaging apparatus transmits the first acoustic radiation force impulses to the external region of the lesion.

For example, as shown in FIG. 4, the rest in the target tissue other than the region surrounded by dotted line is the external region of the lesion. When calculating the shear wave elasticity result for the external region of the lesion, it is assumed that point A is the point to which the first acoustic radiation force impulses are transmitted by the ultrasonic imaging apparatus, that is, the first acoustic radiation force impulses generate shear waves at point A, and point A is the location of the shear wave source. After generating the shear waves at point A, the shear waves may propagate to both sides and pass through point B. The ultrasonic imaging apparatus may then transmit a series of ultrasonic waves to point B to track the shear waves propagating in the external region of the lesion, receive the echo signals of the ultrasonic waves, determine the duration t of the propagation of the shear waves from point A to point B according to the echo signals, and calculate the propagation velocity of the shear waves (that is the propagation velocity of the shear waves passing through point B, i.e. Vs=l/t) according to the distance l between point A and point B and the duration t.

In addition, another way to calculate the propagation velocity of the shear waves may also be to determine the time interval between the propagation of the first shear waves to the first reference position and to the second reference position according to the first ultrasonic echo signals from the first reference position in the external region of the lesion and the first ultrasonic echo signals from the second reference position in the external region of the lesion, and determine the propagation velocity of the first shear waves in the external region of the lesion according to the distance between the first reference position and the second reference position and the time interval.

Again taking FIG. 4 as an example, after generating shear waves at point A, the shear waves may propagate to both sides and pass through points B and C. The ultrasonic imaging apparatus may transmit successively a series of ultrasonic waves to point B and point C to track the shear waves propagating in the external region of the lesion, and receive the echo signals of the ultrasonic waves at point B and that at point C. The time of the shear waves propagating to point B may be determined according to the echo signals of the ultrasonic waves at point B, and the time of the shear waves propagating to point C may be determined according to the echo signals of the ultrasonic waves at point C; in this respect, a time interval t between these two times may be determined, and according to the distance l between point B and point C and the duration t, the propagation velocity of the shear waves may be calculated, that is the propagation velocity of the shear waves is Vs=l/t.

For the above two ways to calculate the propagation velocity of the shear waves, the determination of the shear waves propagating to the first reference position or to the second reference position may be done by, according to the tissue displacement-tissue velocity curve at the reference position, finding the time when the shear waves propagating to the reference position. For example, when the tissue displacement at the reference position reaches its peak, it is considered that the shear waves have just reached the reference position.

After obtaining the propagation velocity of shear waves, various physical quantities reflecting the hardness of the tissue may be further calculated based on formulas such as Young's modulus E=3*ρVs² or shear modulus G=ρVs², where ρ refers to the density of the tissue. The higher the propagation velocity Vs of the shear waves, or the higher the Young's modulus E, or the higher the shear modulus G, the higher the hardness of the tissue.

Therefore, the first shear wave elasticity result may include the propagation velocity of the first shear waves, or the first Young's modulus obtained by the conversion of the propagation velocity of the first shear waves, or the first shear modulus obtained by the conversion of the propagation velocity of the first shear waves; then the quantitative elasticity result may include the propagation velocity of the second shear waves propagating in the internal region of the lesion, or the second Young's modulus obtained by the conversion of the propagation velocity of the second shear waves, or the second shear modulus obtained by the conversion of the propagation velocity of the second shear waves.

In some preferred embodiments, the ultrasonic imaging apparatus may display the ultrasonic image, as well as the first strain elasticity result, the first shear wave elasticity result and the second strain elasticity result, and the quantitative elasticity result, so as to facilitate users to compare the hardness of the external region of the lesion with that of the internal region of the lesion, as well as the difference in the internal region of the lesion between the hardness reflected by the strain elasticity result and the hardness reflected by the quantitative elasticity result.

Alternatively, the ultrasonic imaging apparatus may display the ultrasonic image, as well as the first shear wave elasticity result and the quantitative elasticity result, so as to facilitate users to compare the hardness of the external region with that of the internal region of the lesion. Alternatively, in addition to displaying the ultrasonic image, the first shear wave elasticity result and the quantitative elasticity result, a comparison result between the first shear wave elasticity result and the quantitative elasticity result may also be displayed, so that the difference between the hardness of the external region and that of the internal region of the lesion can be determined directly according to the comparison result.

The comparison result may be the difference or ratio between the first shear wave elasticity result and the quantitative elasticity result, so that the difference in tissue elasticity or tissue hardness between the internal region of the lesion and the external region of the lesion can be known according to the comparison result.

In some preferred embodiments, the way to determine the external region of the lesion of the target tissue according to the ultrasonic image may be to determine the location of the first region of interest in the ultrasonic image, take the first region of interest as the external region of the lesion, and mark the first region of interest in a first mark pattern on the ultrasonic image according to the location of the first region of interest; wherein the first region of interest may include a diffuse disease region or a non-diseased region.

When determining the internal region of the lesion of the target tissue according to the ultrasonic image, the location of the second region of interest in the ultrasonic image may be determined; and the second region of interest may be taken as the internal region of the lesion, and be marked in a second mark pattern different from the first mark pattern on the ultrasonic image according to the location of the second region of interest. The second region of interest may include a focal disease region. The depth of the diffuse disease region or the non-diseased region relative to a body surface may be less than the depth of the focal disease region relative to the body surface.

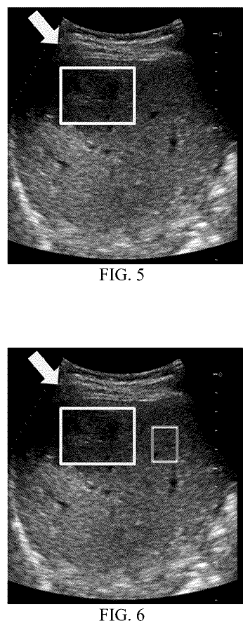

Due to the different processing operations for signal transmission and reception in the internal and external regions of the lesion, it is necessary to differentiate the internal region of the lesion from the external region of the lesion to perform different processing operations. Prior to this, the target tissue of the object under examination may be performed with ultrasound scanning under a scanning operation to obtain an ultrasonic image which may be of B-type, D-type, M-type and other types. After obtaining the ultrasonic image, the ultrasonic imaging apparatus may determine a region of interest containing the focal disease region in the ultrasonic image, take the region of interest as the internal region of the lesion, and mark its location. As shown in FIG. 5, after obtaining the ultrasonic image, the region of interest thereof may be determined as a closed region containing the lesion, be taken as the internal region of the lesion, and be marked by a rectangular box to indicate its location (such as the rectangular box pointed by the arrow). Of course, the region of interest may also be shown in a circle, an oval, or a box representing the boundary of a lesion drawn on the 2D image. After determining the internal region of the lesion, the region outside the closed region where the lesion is located is the external region of the lesion, and then different processing operations may be performed separately on the internal and external regions of the lesion.

After marking the rectangular box corresponding to the internal region of the lesion in the example shown in FIG. 5, the region of interest containing a diffuse disease region or a non-diseased region in the ultrasonic image may be further determined as the external region of the lesion, and the location of the external region of the lesion may be marked in the ultrasonic image, that is, the closed region shown by the right rectangular box in FIG. 6. The rectangular box on the right and that on the left in the figure may be different in mark patterns; for example, the border of the box on the left and that on the right may be displayed in different colors, or in different line shapes (such as solid and dashed lines), or in lines with different thicknesses, and so on. Accordingly, the internal region of the lesion can be differentiated from the external region of the lesion in the ultrasonic image by boxes with different mark patterns.

In some preferred embodiments, when calculating the quantitative elasticity result for the internal region of the lesion, a stress result may be determined according to the first strain elasticity result and the first shear wave elasticity result, and a quantitative elasticity result corresponding to the second strain elasticity result may be determined according to the stress result.

For example, as mentioned above, for the formula OutStrain*OutE=Instrain(i)*Ink(i), the product of the first strain elasticity result OutStrain and the first shear wave elasticity result OutE is the stress to which the external region of the lesion is subjected, and the quantitative elasticity result for the internal region of the lesion is InE(i)=OutStrain*OutE/Instrain(i).

In some embodiments, the first ultrasonic waves for detecting the changes in displacement or strain of the tissue in the external region of the lesion and tracking the first shear waves propagating in the external region of the lesion may include a plurality of sets of ultrasonic waves, wherein the first ultrasonic echo signals corresponding to the one set of ultrasonic waves may be configured to be processed to obtain the first strain elasticity result, and the first ultrasonic echo signals corresponding to another set of ultrasonic waves may be configured to be processed to obtain the first shear wave elasticity result. That is, the first shear wave elasticity result and the first strain elasticity result for the external region of the lesion may be obtained by the process of the ultrasonic echo signals corresponding to different sets of ultrasonic waves.

In addition, the first shear wave elasticity result and the first strain elasticity result for the external region of the lesion may be obtained by the process of the ultrasonic echo signals corresponding to the same set of the ultrasonic waves. In other words, the first ultrasonic waves may include one set of ultrasonic waves, the first strain elasticity result may be obtained by the process of the first ultrasonic echo signals corresponding to the one set of ultrasonic waves, and the first shear wave elasticity result may also be obtained by the process of the first ultrasonic echo signals corresponding to the one set of ultrasonic waves.

In some embodiments, the second acoustic radiation force impulses transmitted to the internal region of the lesion may also generate second shear waves propagating in the internal region of the lesion; accordingly, a shear wave elasticity result for the internal region of the lesion may be calculated according to the second shear waves. That is, the ultrasonic imaging apparatus may transmit third ultrasonic waves to the internal region of the lesion so as to track the second shear waves propagating in the internal region of the lesion, receive the echoes of the third ultrasonic waves to obtain third ultrasonic echo signals, and obtain a second shear wave elasticity result for the internal region of the lesion according to the third ultrasonic echo signals.

After obtaining the second shear wave elasticity result for the internal region of the lesion, the ultrasonic imaging apparatus may also compare and display the second shear wave elasticity result and the quantitative elasticity result, so that the result obtained by conventional shear wave elasticity measurement and the result obtained by the aforesaid quantitative calculation can be compared and the difference therebetween can be provided to users.

Alternatively, a final elasticity result may be determined according to the second shear wave elasticity result and the quantitative elasticity result. For example, a weighted sum of the second shear wave elasticity result and the quantitative elasticity result may be calculated (the weights of each can be preset by users) as the final elasticity result for the internal region of the lesion, and the final elasticity result may be displayed at a location corresponding to the second shear wave elasticity result.

Accordingly, in some embodiments and preferred implementations thereof, the quantitative elasticity result for the internal region of the lesion can be obtained by the calculation according to the shear wave elasticity results and the strain elasticity results for the external region of the lesion and the strain elasticity results for the internal region of the lesion. The quantitative elasticity result may be a specific numerical value, which can quantitatively determine the elasticity and hardness of the tissue in the internal region of the lesion. Moreover, since the shear wave elasticity results and strain elasticity results for the external region of the lesion and the strain elasticity results for the internal region of the lesion are accurate and reliable, compared with directly measuring the shear wave elasticity of the internal region of the lesion, the quantitative elasticity results for the internal region of the lesion can be calculated more accurately in some embodiments, thereby better describing the morphology and boundary of the lesion.

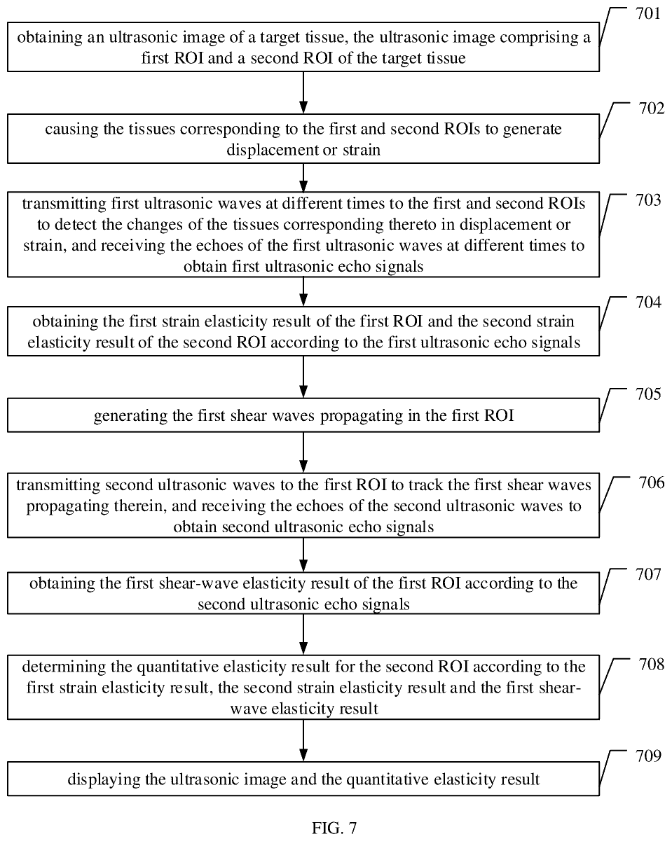

Based on the same inventive concept as the embodiment shown in FIG. 2, an ultrasonic elasticity imaging method may be provided in another embodiment of the present disclosure. Please refer to FIG. 7, the ultrasonic elasticity imaging method in another embodiment of the present disclosure may include the following steps:
Step 701: obtaining an ultrasonic image of a target tissue, the ultrasonic image comprising a first region of interest and a second region of interest of the target tissue;

The ultrasonic imaging apparatus may perform ultrasound scanning on the target tissue of an object under examination under user operation to obtain the ultrasonic image of the target tissue. The first region of interest and the second region of interest of the target tissue in the ultrasonic image may be determined automatically by the ultrasonic imaging apparatus, or be set by users. The ultrasonic imaging apparatus may determine the first region of interest and the second region of interest according to operations set by users. The way in which the ultrasonic imaging apparatus determines the region of interest is not limited in above embodiments.

The target tissue may be any organ tissue of the object under examination, such as a liver tissue. The object under examination referred to may be human body or various animals. When the target tissue is a liver tissue, the first region of interest may include a diffuse disease region outside the lesion or a non-diseased region outside the lesion of the liver tissue, and the second region of interest may include a focal disease region inside the lesion of the liver tissue. The focal disease region inside the lesion may restrict the propagation of the shear waves, leading to inaccurate results obtained by conventional shear wave elasticity measurement; and the diffuse disease region outside the lesion or the non-diseased region outside the lesion has little influence on the penetration of the shear waves, so the results obtained by conventional shear wave elasticity measurement are relatively accurate.
Step 702: causing the tissues corresponding to the first and second regions of interest to generate displacement or strain;

The way in which the ultrasonic imaging apparatus makes the tissues corresponding to the first and second regions of interest to generate displacement or strain may be to transmit to the tissues corresponding to the first and second regions of interest with ultrasonic waves which may induce the tissues to vibrate to cause the tissues to generate displacement or strain.
Step 703: transmitting first ultrasonic waves at different times to the first and second regions of interest to detect the changes in the displacement or strain of the tissues corresponding to the first and second regions of interest, and receiving the echoes of the first ultrasonic waves at different times to obtain first ultrasonic echo signals;
Step 704: obtaining the first strain elasticity result for the first region of interest and the second strain elasticity result for the second region of interest according to the first ultrasonic echo signals;

While causing the tissues corresponding to the first and second regions of interest to generate displacement or strain, the ultrasonic imaging apparatus may also transmit the first ultrasonic waves at different times to the first and second regions of interest to detect changes in displacement or strain of the tissues corresponding to the first and second regions of interest. Moreover, the first strain elasticity result for the first region of interest and the second strain elasticity result for the second region of interest may be obtained according to the first ultrasonic echo signals corresponding to the first ultrasonic waves at different times.
Step 705: generating the first shear waves propagating in the first region of interest;
Step 706: transmitting second ultrasonic waves to the first region of interest to track the first shear waves propagating in the first region of interest, and receiving the echoes of the second ultrasonic waves to obtain second ultrasonic echo signals;
Step 707: obtaining the first shear wave elasticity result for the first region of interest according to the second ultrasonic echo signals;

The ultrasonic imaging apparatus may also generate the first shear waves propagating in the first region of interest, transmit the second ultrasonic waves for tracking the first shear waves, and obtain the first shear wave elasticity result for the first region of interest according to the second ultrasonic echo signals corresponding to the second ultrasonic waves.
Step 708: determining the quantitative elasticity result for the second region of interest according to the first strain elasticity result, the second strain elasticity result and the first shear wave elasticity result; and
Step 709: displaying the ultrasonic image and the quantitative elasticity result.

The ultrasonic imaging apparatus may determine the quantitative elasticity result for the second region of interest according to the first strain elasticity result, the second strain elasticity result and the first shear wave elasticity result, and display the ultrasonic image and the quantitative elasticity result for the second region of interest thereof.

In some embodiments, the ultrasonic imaging apparatus may obtain the first shear wave elasticity result and the first strain elasticity result for the first region of interest in the target tissue of the object under examination respectively, obtain the second strain elasticity result for the second region of interest in the target tissue, and determine the quantitative elasticity result for the second region of interest according to the first shear wave elasticity result, the first strain elasticity result and the second strain elasticity result. Since the shear wave elasticity result and the strain elasticity result for the first region of interest and the strain elasticity result for the second region of interest are accurate, the quantitative elasticity result for the second region of interest calculated from the above results is more accurate than the result obtained by directly measuring the shear wave elasticity of second region of interest, and the morphology and boundary of the lesion can be presented more accurately.

In some embodiments, there may be one or more target tissues; and the tissues corresponding to the first and second regions of interest may be different regions of the same target tissue, or the tissues corresponding to the first and second regions of interest may be different target tissues.

For example, the first region of interest may be a diffuse disease region outside the lesion or a non-diseased region outside the lesion of the liver tissue, and the second region of interest may be a focal disease region inside the lesion of the liver tissue. Alternatively, the first region of interest may be a diffuse disease region outside the lesion or a non-diseased region outside the lesion of spleen tissue, and the second region of interest may be a focal disease region inside the lesion of the liver tissue.

Due to the fact that the deeper the location relative to the body surface in the tissue, the less conducive it is to the propagation of shear waves; accordingly, the shear wave elasticity results obtained by conventional shear wave elasticity measurements at that location are not accurate. In this respect, in some embodiments, the depth of the tissue region corresponding to the first region of interest relative to the body surface may be less than the depth of the tissue region corresponding to the second region of interest relative to the body surface, and the quantitative elasticity result for the second region of interest may be calculated according to the method of the present disclosure, which is more accurate than the result by measuring the deeper second region of interest by conventional shear wave elasticity measurements.

In addition, the greater the hardness of the tissue, the less conducive it is to the propagation of shear waves; accordingly, the results obtained by conventional shear wave elasticity measurements are not accurate. In this respect, in some embodiments, the hardness of the tissue region corresponding to the first region of interest may be less than the hardness of the tissue region corresponding to the second region of interest, and the quantitative elasticity result for the second region of interest may be calculated according to the method of the present disclosure, which is more accurate than the result by measuring the second region of interest with higher hardness by conventional shear wave elasticity measurements.

In some embodiments, the driving forces causing the tissues corresponding to the first and second regions of interest to generate displacement or strain may be the same. The driving forces may be acoustic radiation forces which may be generated by transmitting acoustic radiation force impulses to the first region of interest or the second region of interest. In other words, the acoustic radiation force generated by transmitting the acoustic radiation force impulses to the first region of interest may be the same as the acoustic radiation force generated by transmitting the acoustic radiation force impulses to the second region of interest. In addition, the emission of the acoustic radiation force impulses to the first region of interest may also generate the first shear waves propagating in the first region of interest, and thus the first shear wave elasticity result for the first region of interest may be obtained according to the first shear waves.

Based on this, according to the stress formula Stress=Strain*E, under the same or approximately the same stress, that is, the stress OutStress at the first region of interest is equal to the stress InSstress at the second region of interest, and then according to the formula Stress=Strain*E, it can be obtain:

$$OutStrain * OutE = Instrain ( i ) * InE ( i ) ;$$

where OutStrain refers to the first strain elasticity result, OutE refers to the first shear wave elasticity result, OutStrain*OutE refers to the stress OutStress at the first region of interest; Instrain(i) refers to the second strain elasticity result, Ink(i) refers to the quantitative elasticity result, and Instrain(i)*InE(i) refers to the stress InStress of the second region of interest.

Accordingly, the quantitative elasticity result for the second region of interest may be calculated according to the above principle.

In some embodiments, the display of the ultrasonic image and the quantitative elasticity result may include: displaying the ultrasonic image, mapping the quantitative elasticity result for the second region of interest into a quantitative elasticity image with a target display effect, and displaying the quantitative elasticity image with the target display effect. The target display effect may include any one or more of display effects of grayscale, pseudo-color, and color.

In some embodiments, the ultrasonic imaging apparatus may display the ultrasonic image, as well as the first strain elasticity result, the first shear wave elasticity result and the second strain elasticity result, and the quantitative elasticity result, so as to facilitate users to compare the hardness of the first region of interest with that of the second region of interest, as well as the difference in the second region of interest between the hardness reflected by the strain elasticity result and the hardness reflected by the quantitative elasticity result.

Alternatively, the ultrasonic imaging apparatus may display the ultrasonic image, as well as the first shear wave elasticity result and the quantitative elasticity result, so as to facilitate users to compare the hardness of the first region of interest with that of the second region of interest. Alternatively, in addition to displaying the ultrasonic image, the first shear wave elasticity result and the quantitative elasticity result, a comparison result between the first shear wave elasticity result and the quantitative elasticity result may also be displayed. The difference between the hardness of the first region of interest and that of the second region of interest can be determined directly according to the comparison result.

The comparison result may be the difference or ratio between the first shear wave elasticity result and the quantitative elasticity result, so that users can know the difference in tissue elasticity or tissue hardness between the internal region of the lesion and the external region of the lesion according to the comparison result.

In some embodiments, the ultrasonic imaging apparatus may mark the first region of interest in a first mark pattern on the ultrasonic image according to the location of the first region of interest, and mark the second region of interest in a second mark pattern different from the first mark pattern on the ultrasonic image according to the location of the second region of interest.

The first mark pattern or the second mark pattern may be any closed graphic; for example, the region of interest may also be marked in a circle, an oval, or a box representing the boundary of a lesion drawn on the 2D image. Different mark patterns may be boxes displayed in different colors, or in different line shapes (such as solid and dashed lines), or in lines with different thicknesses, and so on. Accordingly, the first region of interest can be differentiated from the second region of interest in the ultrasonic image by patterns with different mark patterns.

In some embodiments, when calculating the quantitative elasticity result for the second region of interest, a stress result may be determined according to the first strain elasticity result and the first shear wave elasticity result, and a quantitative elasticity result corresponding to the second strain elasticity result may be determined according to the stress result.

For example, as mentioned above, for the formula OutStrain*OutE=Instrain(i)*InE(i), the product of the first strain elasticity result OutStrain and the first shear wave elasticity result OutE is the stress to which the external region of the lesion is subjected, and the quantitative elasticity result for the second region of interest is InE(i)=OutStrain*OutE/Instrain(i).

In some embodiments, the emission of the acoustic radiation force impulses to the second region of interest may also generate the second shear waves propagating in the second region of interest; accordingly, the shear wave elasticity result for the second region of interest may be calculated according to the second shear waves. That is, the ultrasonic imaging apparatus may transmit third ultrasonic waves to the second region of interest to track the second shear waves propagating in the second region of interest, receive the echoes of the third ultrasonic waves to obtain the third ultrasonic echo signals, and obtain the second shear wave elasticity result for the second region of interest according to the third ultrasonic echo signals.

After obtaining the second shear wave elasticity result for the second region of interest, the ultrasonic imaging apparatus may also compare and display the second shear wave elasticity result and the quantitative elasticity result, so that the result obtained by conventional shear wave elasticity measurement and the result obtained by the aforesaid quantitative calculation can be compared and the difference therebetween can be provided to users.

Alternatively, a final elasticity result may be determined according to the second shear wave elasticity result and the quantitative elasticity result. For example, a weighted sum of the second shear wave elasticity result and the quantitative elasticity result may be calculated (the weights of each can be preset by users) as the final elasticity result for the internal region of the lesion, and the final elasticity result may be displayed at a position corresponding to the second shear wave elasticity result.

Below, based on the specific structural components and functions of the ultrasonic imaging apparatus shown in FIG. 1, the various components of the ultrasonic imaging apparatus, as well as the functions and operations performed by each component thereof will be further explained in detailed.

In some embodiments, the ultrasonic imaging apparatus may include:
an ultrasonic probe;
a transmitting circuit for exciting the ultrasonic probe to transmit ultrasonic waves to a target tissue of an object under examination;
a receiving circuit for controlling the ultrasonic probe to receive echoes of the ultrasonic waves from the target tissue to obtain ultrasonic echo signals; and
a processor for processing the ultrasonic echo signals to obtain an ultrasonic image of the target tissue and, according to the ultrasonic image, performing the ultrasonic elasticity imaging method executed by the ultrasonic imaging apparatus in the above embodiments shown in FIG. 2 or FIG. 7 and the implementations thereof.

The functions and operations performed by the components of the ultrasonic imaging apparatus in some embodiment are similar to those performed by the ultrasonic imaging apparatus in the embodiments shown in FIG. 2 or FIG. 7 above and their multiple preferred implementations, and will not be repeated here.

In some embodiments, the ultrasonic imaging apparatus can obtain a first shear wave elasticity result and a first strain elasticity result for the external region of a lesion in the target tissue of an object under examination, obtain a second strain elasticity result for the internal region of the lesion in the target tissue, and determine a quantitative elasticity result for the internal region of the lesion according to the first shear wave elasticity result, the first strain elasticity result and the second strain elasticity result. Since the shear wave elasticity result and the strain elasticity result for the external region of the lesion and the strain elasticity result for the internal region of the lesion are accurate, the quantitative elasticity result for the internal region of the lesion calculated from the above results is more accurate than the result obtained by directly measuring the shear wave elasticity of the internal region of the lesion, and the morphology and boundary of the lesion can be presented more accurately.

Those skilled in the art can clearly understand that, for the sake of convenience and simplicity in description, the specific working process of the system, device and unit described above can refer to the corresponding process in the above-mentioned embodiments of the method, and will not be repeated here.

In a plurality of embodiments provided in the present disclosure, it should be understood that the disclosed systems, apparatus, and methods may be implemented in other ways. For example, the device embodiments described above are only illustrative. For example, the division of units is only a logical functional division. In practical implementation, there may be other division methods, such as multiple units or components being combined or integrated into another system, or some features being ignored or not executed. In addition, the coupling or direct coupling or communication connection displayed or discussed may be indirect coupling or communication connection through some interface, device or unit, which may be electrical, mechanical or other forms.

The units described as separate components may or may not be physically separate, and the components displayed as units may or may not be physical units, that is, they may be located in one place, or may be distributed across multiple network units. Some or all of the units may be selected according to the actual needs to achieve the purpose of the embodiments.

In addition, the functional units in various embodiments of the present disclosure may be integrated in a single processing unit, or the units may exist separately physically, or two or more units may be integrated in a single unit. The integrated unit mentioned above may be realized either in the form of hardware or in the form of software functional unit.

The integrated unit, if implemented in the form of software functional units and marketed or used as an independent product, may be stored in a computer readable storage medium. Based on this understanding, the technical solution of the present disclosure may be substantially or partially contributed to the prior art or all or part of the technical solution may be embodied in the form of a software product, which is stored in a storage medium and includes a number of instructions for making a computer device (which may be a personal computer, a server, or a network device, etc.) perform all or part of the steps of the method described in various embodiments of the present disclosure. The aforementioned storage media may include: USB disk, mobile hard disk, read-only memory (ROM), random access memory (RAM), disk or disc and other media that can store program code.

What is claimed is:

1. An ultrasonic elasticity imaging method, comprising:
obtaining an ultrasonic image of a target tissue, and determining an internal region of a lesion and an external region of the lesion of the target tissue according to the ultrasonic image;
transmitting first acoustic radiation force impulses to the external region of the lesion, so as to cause a tissue in the external region of the lesion to generate first displacement or strain and generate first shear waves propagating in the external region of the lesion;
transmitting first ultrasonic waves at different times to the external region of the lesion for detecting changes in the first displacement or strain of the tissue in the external region of the lesion and tracking the first shear waves propagating in the external region of the lesion, and receiving echoes of the first ultrasonic waves to obtain first ultrasonic echo signals;
obtaining a first strain elasticity result for the external region of the lesion according to the first ultrasonic echo signals;
obtaining a first shear wave elasticity result for the external region of the lesion according to the first ultrasonic echo signals;
transmitting second acoustic radiation force impulses to the internal region of the lesion, so as to cause a tissue in the internal region of the lesion to generate second displacement or strain;
transmitting second ultrasonic waves at different times to the internal region of the lesion for detecting changes in the second displacement or strain of the tissue in the internal region of the lesion, and receiving echoes of the second ultrasonic waves to obtain second ultrasonic echo signals;
obtaining a second strain elasticity result for the internal region of the lesion according to the second ultrasonic echo signals;
obtaining a quantitative elasticity result for the internal region of the lesion according to the first strain elasticity result, the first shear wave elasticity result and the second strain elasticity result; and
displaying the ultrasonic image and the quantitative elasticity result.

2. The method of claim 1, wherein an acoustic radiation force generated by the first acoustic radiation force impulses on a first target position in the external region of the lesion and an acoustic radiation force generated by the second acoustic radiation force impulses on a second target position in the internal region of the lesion are identical, or a difference therebetween is within a predetermined range;
wherein the first target position is a position corresponding to the first strain elasticity result, and the second target position is a position corresponding to the second strain elasticity result.

3. The method of claim 2, wherein a value of a parameter of the first acoustic radiation force impulses are identical to a value of a parameter of the second acoustic radiation force impulses, and a depth of the first target position in the external region of the lesion is identical to a depth of the second target position in the internal region of the lesion.

4. The method of claim 2, wherein
transmitting first acoustic radiation force impulses to the external region of the lesion comprises: transmitting the first acoustic radiation force impulses with a parameter value thereof being a first target value to the external region of the lesion; and
transmitting second acoustic radiation force impulses to the internal region of the lesion comprises: transmitting the second acoustic radiation force impulses with a parameter value thereof being a second target value to the internal region of the lesion;
wherein the second target value is determined according to a depth of the first target position in the external region of the lesion, a depth of the second target position in the internal region of the lesion, and the first target value.

5. The method of claim 3, wherein the parameter of the first acoustic radiation force impulses and/or the parameter of the second acoustic radiation force impulses comprises at least one of a pulse length, a pulse waveform, an emission frequency, an emission aperture and a focusing intensity.

6. The method of claim 1, wherein
transmitting second acoustic radiation force impulses to the internal region of the lesion comprises: transmitting the second acoustic radiation force impulses to one or more target positions in the internal region of the lesion, so as to cause tissue(s) corresponding to the one or more target positions to generate the second displacement or strain;
transmitting second ultrasonic waves at different times to the internal region of the lesion comprises: transmitting the second ultrasonic waves at different times to the one or more target positions in the internal region of the lesion so as to detect changes in the second displacement or strain of the tissue(s) corresponding to the one or more target positions, and receiving echoes of the second ultrasonic waves to obtain second ultrasonic echo signals comprises: receiving the echoes of the second ultrasonic waves from the one or more target positions to obtain the second ultrasonic echo signals;
obtaining a second strain elasticity result for the internal region of the lesion according to the second ultrasonic echo signals comprises: obtaining the second strain elasticity result for the one or more target positions according to the second ultrasonic echo signals from the one or more target positions; and
determining a quantitative elasticity result for the internal region of the lesion according to the first strain elasticity result, the first shear wave elasticity result and the second strain elasticity result comprises: determining the quantitative elasticity result for the one or more target positions in the internal region of the lesion according to the first strain elasticity result, the first shear wave elasticity result, and the second strain elasticity result for the one or more target positions in the internal region of the lesion.

7. The method of claim 6, after obtaining the quantitative elasticity result for the target positions in the internal region of the lesion, further comprising:
making statistics on the quantitative elasticity result for the target positions in the internal region of the lesion, and displaying a statistical result therefrom, the statistical result including one or more of a median, a mean, a maximum, a minimum, and a standard deviation.

8. The method of claim 6, wherein displaying the ultrasonic image and the quantitative elasticity result comprises:
displaying the ultrasonic image, mapping the quantitative elasticity result for the target positions in the internal region of the lesion into a quantitative elasticity image with a target display effect, and displaying the quantitative elasticity image with the target display effect, the target display effect including any one or more of grayscale, pseudo-color, and color.

9. The method of claim 1, wherein the target tissue comprises a liver tissue, the external region of the lesion comprises a diffuse disease region outside the lesion or a non-diseased region outside the lesion of the liver tissue, and the internal region of the lesion comprises a focal disease region inside the lesion of the liver tissue.

10. The method of claim 1, wherein the first shear wave elasticity result comprises a propagation velocity of the first shear waves, a first Young's modulus or a first shear modulus, and the quantitative elasticity result comprises a propagation velocity of second shear waves, a second Young's modulus, or a second shear modulus.

11. The method of claim 1, wherein
obtaining a first shear wave elasticity result for the external region of the lesion according to the first ultrasonic echo signals comprises:
determining a duration of the first shear waves propagating from a wave source position to a first reference position according to the first ultrasonic echo signals from the first reference position in the external region of the lesion; and
determining a propagation velocity of the first shear waves in the external region of the lesion according to a distance between the wave source position of the first shear waves and the first reference position and the duration therebetween; or,
obtaining a first shear wave elasticity result for the external region of the lesion according to the first ultrasonic echo signals comprises:
determining a time interval between the first shear waves propagating to a first reference position and to a second reference position according to the first ultrasonic echo signals from the first reference position in the external region of the lesion and the first ultrasonic echo signals from the second reference position in the external region of the lesion; and
determining a propagation velocity of the first shear waves in the external region of the lesion according to a distance between the first reference position and the second reference position and the time interval.

12. The method of claim 1, further comprising:
displaying the first strain elasticity result, the first shear wave elasticity result and the second strain elasticity result; or
displaying the first shear wave elasticity result; or
displaying the first shear wave elasticity result, and displaying a comparison result between the first shear wave elasticity result and the quantitative elasticity result.

13. The method of claim 1, wherein determining the external region of the lesion of the target tissue according to the ultrasonic image comprises:
determining a location of a first region of interest in the ultrasonic image, taking the first region of interest as the external region of the lesion, and marking the first region of interest in a first mark pattern on the ultrasonic image according to the location of the first region of interest, the first region of interest including a diffuse disease region or a non-diseased region.

14. The method of claim 13, wherein determining the internal region of the lesion of the target tissue according to ultrasonic image comprises:
determining a location of a second region of interest in the ultrasonic image, taking the second region of interest as the internal region of the lesion, and marking the second region of interest in a second mark pattern different from the first mark pattern on the ultrasonic image according to the location of the second region of interest, the second region of interest including a focal disease region, and a depth of the diffuse disease region or the non-diseased region relative to a body surface being less than a depth of the focal disease region relative to the body surface.

15. The method of claim 1, wherein determining the quantitative elasticity result for the internal region of the lesion according to the first strain elasticity result, the first shear wave elasticity result and the second strain elasticity result comprises:
determining a first stress result according to the first strain elasticity result and the first shear wave elasticity result; and
determining the quantitative elasticity result according to the first stress result and the second strain elasticity result.

16. The method of claim 1, wherein,
the first ultrasonic waves comprise a plurality of sets of ultrasonic waves, the first strain elasticity result is obtained by processing according to the first ultrasonic echo signals corresponding to one set of ultrasonic waves therefrom, and the first shear wave elasticity result is obtained by processing according to the first ultrasonic echo signals corresponding to another set of ultrasonic waves therefrom; or
the first ultrasonic waves comprise one set of ultrasonic waves, the first strain elasticity result is obtained by processing according to the first ultrasonic echo signals corresponding to the one set of ultrasonic waves, and the first shear wave elasticity result is obtained by processing according to the first ultrasonic echo signals corresponding to the one set of ultrasonic waves.

17. The method of claim 1, further comprising:
generating second shear waves propagating in the internal region of the lesion;
transmitting third ultrasonic waves to the internal region of the lesion to track the second shear waves propagating in the internal region of the lesion, and receiving echoes of the third ultrasonic waves to obtain third ultrasonic echo signals;
obtaining a second shear wave elasticity result for the internal region of the lesion according to the third ultrasonic echo signals; and
displaying the second shear wave elasticity result and the quantitative elasticity result, or determining a final elasticity result according to the second shear wave elasticity result and the quantitative elasticity result.

18. An ultrasonic elasticity imaging method, comprising:
obtaining an ultrasonic image of a target tissue, the ultrasonic image comprising a first region of interest and a second region of interest of the target tissue;
causing tissues in the first and second regions of interest to generate displacement or strain;
transmitting first ultrasonic waves at different times to the first and second regions of interest for detecting changes in the displacement or strain of the tissues in the first and second regions of interest, and receiving echoes of the first ultrasonic waves to obtain first ultrasonic echo signals;
obtaining a first strain elasticity result for the first region of interest and a second strain elasticity result for the second region of interest according to the first ultrasonic echo signals;
generating first shear waves propagating in the first region of interest;
transmitting second ultrasonic waves to the first region of interest for tracking the first shear waves propagating in the first region of interest, and receiving echoes of the second ultrasonic waves to obtain second ultrasonic echo signals;
obtaining a first shear wave elasticity result for the first region of interest according to the second ultrasonic echo signals;
determining a quantitative elasticity result for the second region of interest according to the first strain elasticity result, the second strain elasticity result and the first shear wave elasticity result; and
displaying the ultrasonic image and the quantitative elasticity result.

19. The method of claim 18, wherein a depth of a tissue region corresponding to the first region of interest relative to a body surface is less than a depth of a tissue region corresponding to the second region of interest relative to the body surface; or, a hardness of the tissue region corresponding to the first region of interest is less than a hardness of the tissue region corresponding to the second region of interest.

20. An ultrasonic imaging apparatus, comprising:
an ultrasonic probe;
a transmitting circuit configured to excite the ultrasonic probe to transmit ultrasonic waves to a target tissue of an object under examination;
a receiving circuit configured to control the ultrasonic probe to receive echoes of the ultrasonic waves from the target tissue to obtain ultrasonic echo signals;
a display; and
a processor configured to:
process the ultrasonic echo signals to obtain an ultrasonic image of the target tissue, and determine an internal region of a lesion and an external region of the lesion of the target tissue according to the ultrasonic image;
control the transmitting circuit to excite the ultrasonic probe to transmit first acoustic radiation force impulses to the external region of the lesion, so as to cause a tissue in the external region of the lesion to generate first displacement or strain and generate first shear waves propagating in the external region of the lesion;
control the transmitting circuit to excite the ultrasonic probe to transmit first ultrasonic waves at different times to the external region of the lesion for detecting changes in the first displacement or strain of the tissue in the external region of the lesion and tracking the first shear waves propagating in the external region of the lesion, and receive echoes of the first ultrasonic waves to obtain first ultrasonic echo signals;
obtain a first strain elasticity result for the external region of the lesion according to the first ultrasonic echo signals;
obtain a first shear wave elasticity result for the external region of the lesion according to the first ultrasonic echo signals;
control the transmitting circuit to excite the ultrasonic probe to transmit second acoustic radiation force impulses to the internal region of the lesion, so as to cause a tissue in the internal region of the lesion to generate second displacement or strain;
control the transmitting circuit to excite the ultrasonic probe to transmit second ultrasonic waves at different times to the internal region of the lesion for detecting changes in the second displacement or strain of the tissue in the internal region of the lesion, and receive echoes of the second ultrasonic waves to obtain second ultrasonic echo signals;
obtain a second strain elasticity result for the internal region of the lesion according to the second ultrasonic echo signals;
obtain a quantitative elasticity result for the internal region of the lesion according to the first strain elasticity result, the first shear wave elasticity result and the second strain elasticity result; and
control the display to display the ultrasonic image and the quantitative elasticity result.
